(19) United States
(12) Patent Application Publication
(10) Pub. No.: US 2007/0167615 A1
(43) Pub. Date: Jul. 19, 2007

(54) Macrocyclic oligonucleotide labeling reactants and conjugates derived thereof

(75) Inventor: Jari Hovinen, Raisio (FI)

Correspondence Address: BUCHANAN, INGERSOLL & ROONEY PC, POST OFFICE BOX 1404, ALEXANDRIA, VA, 22313-1404, US

(73) Assignee: WALLAC OY, Turku (FI)

(21) Appl. No.: 11/643,867

(22) Filed: Dec. 22, 2006

Related U.S. Application Data
(60) Provisional application No. 60/754,204, filed on Dec. 28, 2005.

(30) Foreign Application Priority Data
Dec. 29, 2005 (FI) 20055712

(51) Int. Cl. C07H 19/04; C07H 21/04

(52) U.S. Cl. 534/16; 536/25.32; 536/26.1

(57) ABSTRACT

This invention concerns novel labeling reactants, which are derivatives of macrocyclic chelators and which allow site specific introduction of the ligand of said derivatives to oligonucleotides molecules on solid phase.

### CROSS REFERENCE TO RELATED APPLICATIONS

This application claims priority under 35 U.S.C. §119 to U.S. Provisional Application 60/754,204 filed on Dec. 28, 2005,and to Finnish Patent Application 20055712 filed in Finland on Dec. 29, 2005, the entire contents of which are hereby incorporated by reference in their entireties.

### FIELD

This invention relates to derivatives of macrocyclic chelators which allow site specific introduction of the ligand of said derivatives to oligonucleotides molecules on solid phase.

### BACKGROUND

The publication and other materials used herein to illuminate the background of the invention, and in particular, cases to provide additional details respecting the practice, are incorporated by reference.

Because of their high in vivo and in vitro stability macrocyclic chelators, such as 1,4,7-triazacyclononanetriacetic acid (NOTA), 1,4,7,10-tetraazacyclododecanetetraacetic acid (DOTA), 1,4,8,11 -tetraazacyclotetradecane-1,4,8,1 1-tetraacetic acid (TETA) and their derivatives have been used for complexation with radioisotopes of Ga, Cu, Y, In, Lu and Ac. These radioisotopes have been used in tumor imaging and therapy, while the corresponding Gd chelates, in turn, are suitable in magnetic resonance imaging.

In several applications, covalent conjugation of the macrocyclic chelator to bioactive molecules is required. Most commonly, the isothiocyanato, N-hydroxysuccinimide or maleimide derivatives of the chelate are used in the labeling the target molecules in solution [Lewis, M. R., Raubitschek, A., and Shively, 1994,Bioconjugate Chem., 5, 565; Hnatowich, D. J., Winnard Jr., P., Virzi, M., Fogarazi, T., Sano, T., Smith, C. L., Cantor, C. R., and Rusckowski, M., 1995,J. Nucl. Med, 36, 2306.; Winnard, P., Chang, F., Rusckowski, M., Mardirossian, G., and Hnatowich, D. J., 1997,Nucl. Med. Biol. 24, 425]. Several bifunctional macrocyclic chelators are currently commercially available. Since the labeling reactions are performed in the presence of an excess of an activated label, laborious purification procedures cannot be prevented. Especially, when attachment of several label molecules is needed, purification and characterization of the desired biomolecule conjugate may be extremely difficult. The purification problems can be avoided by performing the labeling reaction on solid phase. Hence, most of the impurities can be removed by washings when the biomolecule conjugate is still anchored to the solid support, and after release to the solution, only one chromatographic purification is needed. Although building blocks for the solid phase introduction of DOTA to synthetic oligopeptides have been reported [Heppeler, A., Froidevaux, S., Mäcke, H. R., Jermann, E., Behe, M., Powell, P., and Hennig, M. 1999, Chem. Eur. J., 5, 1894; Bhorade, R., Weissleder, R., Nakakoshi, T., Moore, A. and Tung, C.-H., 2000,Bioconjugate Chem., 11, 301.; Gallazzi, F., Wang, Y., Jia, F., Shenoy, N., Landon, L. A., Hannink, M., Lever, S. Z. and Lewis, M. R. 2003,Bioconjugate Chem., 14, 1083], corresponding oligonucleotide labeling reactants are not availble. The only macrocyclic chelator reported for machine assisted oligonucelotide synthesis is a cyclam derivative which allows introduction of a ⁶⁴Cu or ^{99m}Tc chelate to 5′-terminus of an synthetic oligonucleotide [Wagner, S., Eisenhut, M., Eritja, R., Oberdorfer, F. 1997, Nucleosides, Nucleotides, 16,1789].

### OBJECTS AND SUMMARY

The main object of the present invention is to provide reactants which allow solid phase introduction of macrocyclic chelators to oligonucleotides using a standard oligonuclotide synthesizer. The bioconjugates thus obtained are highly suitable for magnetic resonance imaging (MRI), positron emission tomography (PET), single positron emission computed tomography (SPECT) as well as target-specific radiopharmaceuticals. The major advantage of the present invention are: (i) synthesis of the building blocks is simple and thus these molecules can be synthesized in large scale; (ii) the blocks can be introduced to the biomolecule structure with standard oligonucleotide synthesizer in high efficiencey using normal procedures; (iii) the position of the label in the oligonucleotide chain is not restricted; (iv) the method allows multilabeling. This is very advantageous in applications where high detection sensitivity is required; (v) since the metal is introduced after the chain assembly is completed, the molecule synthesized can be used in various applications simply by changing the metal; (vi) because of the synthetic strategy the oligonucleotide conjugate is always free from unconjugated chelate. This is extremely important in vivo applications.

Thus, according to one aspect, the present invention concerns a labeling reactant of formula (I) suitable for labeling of an oligonucleotide using solid-phase synthesis 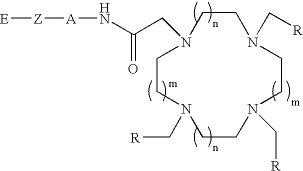
(I)
wherein,
-A- is a linker;
R is —COOR′ or CONHR′ where R′ is an alkyl of 1 to 4 carbon atoms, phenyl or benzyl, which phenyl or benzyl is substituted or unsubstituted;
m and n are independently 0, 1 or 2;
Z is a bridge point and is absent or is a radical of a purine base or a pyrimidine base or a 7-deazapurine base or any other modified base suitable for use in the synthesis of modified oligonucleotides, said base being connected to E via either i) a hydrocarbon chain, which is substituted with a protected hydroxyethyl or hydroxymethyl group, or via ii) a furan ring or pyrane ring or any modified furan or pyrane ring, suitable for use in the synthesis of modified oligonucleotides; and 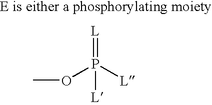
   where L is absent or is O or S;
L′ is H, L′″CH₂CH₂CN or L′″Ar, where Ar is phenyl or its substituted derivative, where the substituent is nitro or chlorine, and L′″ is O or S;
L″ is O⁻, S⁻, Cl, N(i-Pr)₂; or
E is a solid support tethered to Z via a linker arm, which is the same as or different from the linker -A- as defined above.

According to another aspect, the invention concerns an oligonucleotide conjugate synthesized using a oligonucleotide labeling reactant according to this invention.

### DETAILED DESCRIPTION

In case R′ as defined above is a substituted phenyl or substituted benzyl, the preferable substituents are halides, most preferably chloride.

According to a preferable embodiment, the linker -A- is formed from one to ten moieties, each moiety being selected from the group consisting of phenylene, alkyl containing 1-12 carbon atoms, ethynediyl (—C≡C—), ethylenediyl (—C═C—), ether (—O—), thioether (—S—), amide (—CO—NH— and —NH—CO— and —CO—NR″ and —NR″—CO—), carbonyl (—CO—), ester (—COO— and —OOC—), disulfide (—SS—), diaza (—N═N—) and tertiary amine (—NR″—), where R″ represents an alkyl containing less than 5 carbon atoms.

Preferably, the bridge point Z is a radical of any of the bases thymine, uracil, adenine, guanine or cytosine, deazaadenine or deazaguanine and said base is connected to E via either i) a hydrocarbon chain, which is substituted with a protected hydroxyethyl or hydroxymethyl group, or via ii) a furan ring having a protected hydroxymethyl group in its 4-position and optionally a hydroxyl, protected hydroxyl, halogen, most preferably fluorine, or modified hydroxyl group in its 2-position.

According to another preferable embodiment, Z is a radical of adenine, cytosine or 7-deazaadenine where the exocyclic amino group is protected with a protecting group. The protecting group is preferably a benzoyl group. Other preferable protecting groups are, for example isobutyryl, dimethylformamidine, acetyl, t-butylphenoxyacetyl or phenoxyacetyl.

According to another preferable embodiment, Z is a radical of guanine or 7-deazaguanine where the exocyclic amino group is protected with a protecting group. The protecting group is preferably an isobutyryl group, but also other protecting groups can be used, for example dimethylformamidine, t-butylphenoxyacetyl or p-isopropylphenoxyacetyl.

Especially preferable are labeling reactants in which the furan ring in Z is derived from 2-deoxy-D-ribose.

Especially preferable are labeling reactants wherein E-Z-A is selected from the group consisting of the nine structures shown below: 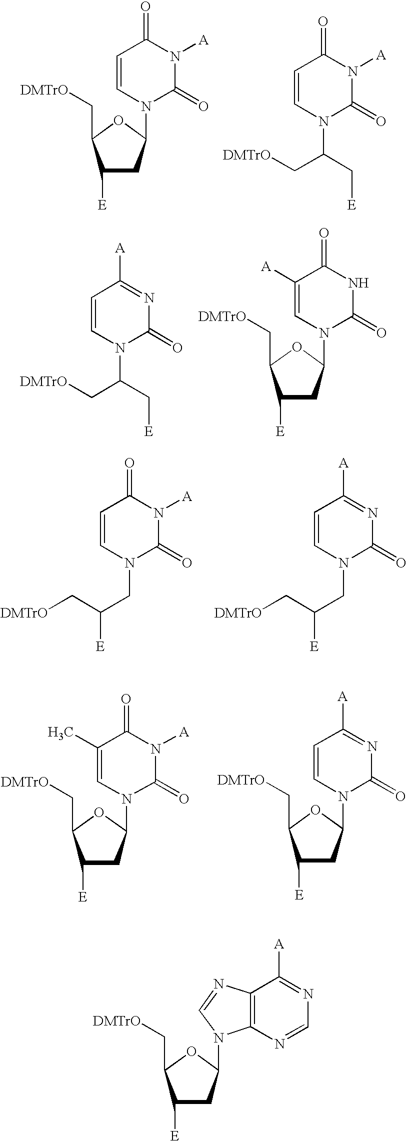
wherein DMTr is dimethoxytrityl.

According to a preferable embodiment, L is absent, L′ is OCH₂CH₂CN and L″ is N(i-Pr)₂.

The chelating agent can be introduced into oligonucleotides with the aid of oligonucleotide synthesizer. A useful method, based on a Mitsonobu alkylation (J Org Chem, 1999, 64, 5083; Nucleosides, Nucleotides, 1999, 18, 1339) is disclosed in U.S. Pat. No. 6,949,696 and U.S. Ser. No. 09/985,454 (AP100695). Said patent publications disclose a method for direct attachment of a desired number of conjugate groups to the oligonucleotide structure during chain assembly. Thus solution phase labeling and laborious purification procedures are avoided. The key reaction in the synthesis strategy towards nucleosidic oligonucleotide building blocks is the aforementioned Mitsunobu alkylation which allows introduction of various chelating agents to the nucleoside, and finally to the oligonucleotide structure. The chelating agents are introduced during the chain assembly. Conversion to the lanthanide chelate takes place after the synthesis during the deprotection steps.

Normal, unmodified oligonucleotides have low stability under physiological conditions because of its degradation by enzymes present in the living cell. It may therefore desirable to create a modified oligonucleotide according to known methods so as to enhance its stability against chemical and enzymatic degradation.

Modifications of oligonucleotides are extensively disclosed in prior art. Reference is made to U.S. Pat. No. 5,612,215. It is known that removal or replacement of the 2′—OH group from the ribose unit in an RNA chain gives a better stability. WO 92/07065 and U.S. Pat. No. 5,672,695 discloses the replacement of the ribose 2′—OH group with halo, amino, azido or sulfhydryl groups. U.S. Pat. No. 5,334,711 discloses the replacement of hydrogen in the 2′—OH group by alkyl or alkenyl, preferably methyl or allyl groups. Furthermore, the internucleotidic phosphodiester linkage can, for example, be modified so that one ore more oxygen is replaced by sulfur, amino, alkyl or alkoxy groups. Preferable modification in the internucleotide linkages are phosphorothioate linkages. Also the base in the nucleotides can be modified.

In some applications it is advantageous that the chelate is neutral. Then, two of the acetate groups can be substituted with amides. Naturally, the stability of these chelates is lower than that of the corresponding acetates.

Experimental Section

The invention is further elucidated by the following non-restricting Examples. The structures and synthetic routes employed in the experimental part are depicted in Scheme 1. Experimental details are given in Examples 1 - 5. Coupling of the oligonucleotide building block to oligonucleotide structure on solid phase, deprotection and convertion to the corresponding gadolinium(III) chelate is given in Example 6.

Procedures

Adsorption column chromatography was performed on columns packed with silica gel 60 (Merck). Reagents for oligonucleotide synthesis were purchased from Proligo. The oligonucleotides were assembled on Applied Biosystems 3400 instrument, using recommended protocols. All dry solvents were from Merck and they were used as received. NMR spectra were recorded on a Brucker 250 spectrometer operating at 250.13 MHz for ¹H and on and on a Jeol LA 400 spectrometer operating at 161.9 MHz for ³¹P. The signal of TMS was used as an internal (¹H) and H₃PO₄ as an external (³¹P) reference. ESI-TOF mass spectra on an Applied Biosystems Mariner instrument.

### EXAMPLES

### Example 1

### The Synthesis of 1,4,7,10-tetraazacyclododecane-4,7,10-tricarboxymethyl methylester-1-carboxymethyl-benzylester, 2.

To a stirred mixture of 1,4,7,10-tetraazacyclododecane-1-carboxymethyl-benzyl ester, 1 (0.45 g, 1.4 mmol), disclosed in Heppler, A. et al., 1999, Chem. Eur. J., 5, 1974, potassium carbonate (0.79 g, 5.7 mmol) in anhydrous acetonitrile (8 mL) was added methyl bromoacetate (0.54 mL, 5.7 mmol, predissolved in 2 mL of MeCN) dropwise during 0.5 h. The reaction was allowed to proceed for an additional 2 h before being filtered. The filtrate was concentrated in vacuo. Purification was performed on silica gel (eluent MeOH: CH₂Cl₂,1:9,v/v). ¹H NMR (CDCl₃): δ 7.35 (5H, m); 5.20 (2H, s); 3.76 (6H, s); 3.74 (3H, s); 3.49-2.35 (24 H). ESI-TOF-MS for C₂₆H₄₁N₄NaO₈⁺ (M+Na)⁺: calcd, 559.27; found, 559.27.

### Example 2

### The Synthesis of 1,4,7,10-tetraazacyclododecane-1,4,7,10-tetraacetic acid tris-methyl ester, 3

Compound 2 (0.23 g, 0.42 mmol) was dissolved in methanol (10 mL). Pd/C (10%, 55 mg) was added and the mixture was hydrogenated at atmospheric pressure overnight. The mixture was filtered through celite and concentrated. ESI-TOF-MS for C₁₉H₃₄N₄NaO₈⁺ (M+Na)⁺: calcd, 469.23; found, 469.22.

### Example 3

### The Synthesis of 2′-deoxy-5′—O—(4,4′-dimethoxytrityl)-3-(6-aminohexyl)uridine, 4.

2′-Deoxy-5′—O—(4,4′-dimethoxytrityl)-3-(6-trifluoroacetamidohexyl)uridine (1.41 g), disclosed in Hovinen, J., Hakala, H., 2001, Org. Lett., 3, 2473, was suspended in the mixture of conc. aqueous ammonia and methanol (1:1,v/v) and heated overnight at reflux. All volatiles were removed in vacuo. The residue was partitioned between water and dichloromethane. The organic layer was dried over Na₂SO₄ and concentrated.

¹H NMR (CDCl₃): δ 7.75 (1H, d, J 8.3, H-6); 7.40-7.23 (9H, DMTr); 6.84 (4H, d, J 8.9, DMTr); 6.31 (1 H, t, J 6.2, H-1′); 5.45 (1H, d, J 8.3, H-5); 4.53 (1 H, m, H-3′); 4.00 (1H, m, H-4′); 3.89 (2H, m); 3.78 (6H, s, 2. OMe); 3.49 (1H, dd, J 10.6 and 3.0, H-5′); 3.41 (1H, dd, J 10.6 and 3.3, H-5″); 2.64 (2H, t, J 6.5); 2.42 (1H, m, H-2″); 2.24 (1H, m, H-2′); 2.19 (3H, br); 1.62 (2H, p, J6.4); 1.40 (2H, p, J 6.7); 1.34 (4H, m). ESI-TOF-MS for C₃₆H₄₄N₃O₇⁺ (M+H)⁺: calcd, 630.31; found, 630.34.

### Example 4

### The synthesis of the DOTA nucleoside, 5.

Compound 3 (0.26 g, 0.58 mmol) and DIPEA (100 μL) were dissolved in dry DMF (9 mL). HATU (220 mg, 0.58 mmol) was added and the mixture was stirred for 30 min at RT. Compound 4 (0.37 g, 0.58 mmol) was added, and the mixture was stirred for 4 h at RT and concentrated. The residue was dissolved in dichoromethane, washed twice with sat. NaHCO₃ and dried. Purification on silica gel (eluent, MeOH: CH₂Cl₂1:9,v/v) gave the title compound. ¹H NMR (CDCl₃) δ7.75 (1H, d, J 8.3,H-6); 7.40-7.22 (9H, DMTr); 6.84 (4H, d, J 8.8); 6.47 (1H, br t, J 4.7, NH); 6.32 (1H, t, J 6.3); 5.43 (1H, d, J 8.0,H-5); 4.59 (1H, m, H-3′); 4.05 (1H, m, H-4′); 3.89 (2H, m); 3.79 (6H, s, 2. OMe); 3.74 (9H, s); 3.42 (2H, d, J 2.9, H-5′ and H-5″); 3.20-2.29 (20H); 1.62 (2H, m); 1.50 (2H, m); 1.35 (4H, m). ESI-TOF-MS for C₅₅H₇₆N₇O₁₄⁺ (M+H)⁺: calcd, 1058.54; found, 1058.54.

### Example 5

### The Synthesis of the phosphoramidite, 6.

Compound 4 (0.30 g, 0.28 mmol) was phosphitylated and purified using the method disclosed in Hovinen, J., Hakala, H., 2001,Org. Lett., 3, 2473. 31P NMR (CDCl₃): δ 149.60 (0.5 P); 149. 20 (0.5 P). ESI-TOF-MS for C₆₄H₉₃N₉O₁₅P⁺ (M+H)⁺: calcd, 1258.65; found, 1258.66.

### Example 6

### The Synthesis and purification of the oligonucleotide conjugates.

A model sequence d(TAA TGT AGC CCC TGA A) was assembled on a 1.0 μmol scale using phosphoramidite chemistry and recommended protocols (DMTr-Off synthesis). Compound 6 was coupled to the 5′-terminus of the oligonucleotide (coupling time 10 min, concentration 0.2 M). As the chain asembly was completed, the oligonucleotides were deprotected and converted to the gadolinium(III) chelate as the following: (i) treatment with 0.1 M NaOH for 4 h at RT (ii) concentration in vacuo in the presence of ammonium chloride (iii) treatment with conc. aqueous ammonia for 16 h at 55° C. (iv) treatment with gadolinium(III) citrate (5 equiv per ligand) for 90 min at RT. Desalting by gel filtration and denaturing PAGE yielded the oligonucleotide conjugate.

It will be appreciated that the methods of the present invention can be incorporated in the form of a variety of embodiments, only a few of which are disclosed herein. It will be apparent for the expert skilled in the field that other embodiments exist and do not depart from the spirit of the invention. Thus, the described embodiments are illustrative and should not be construed as restrictive.

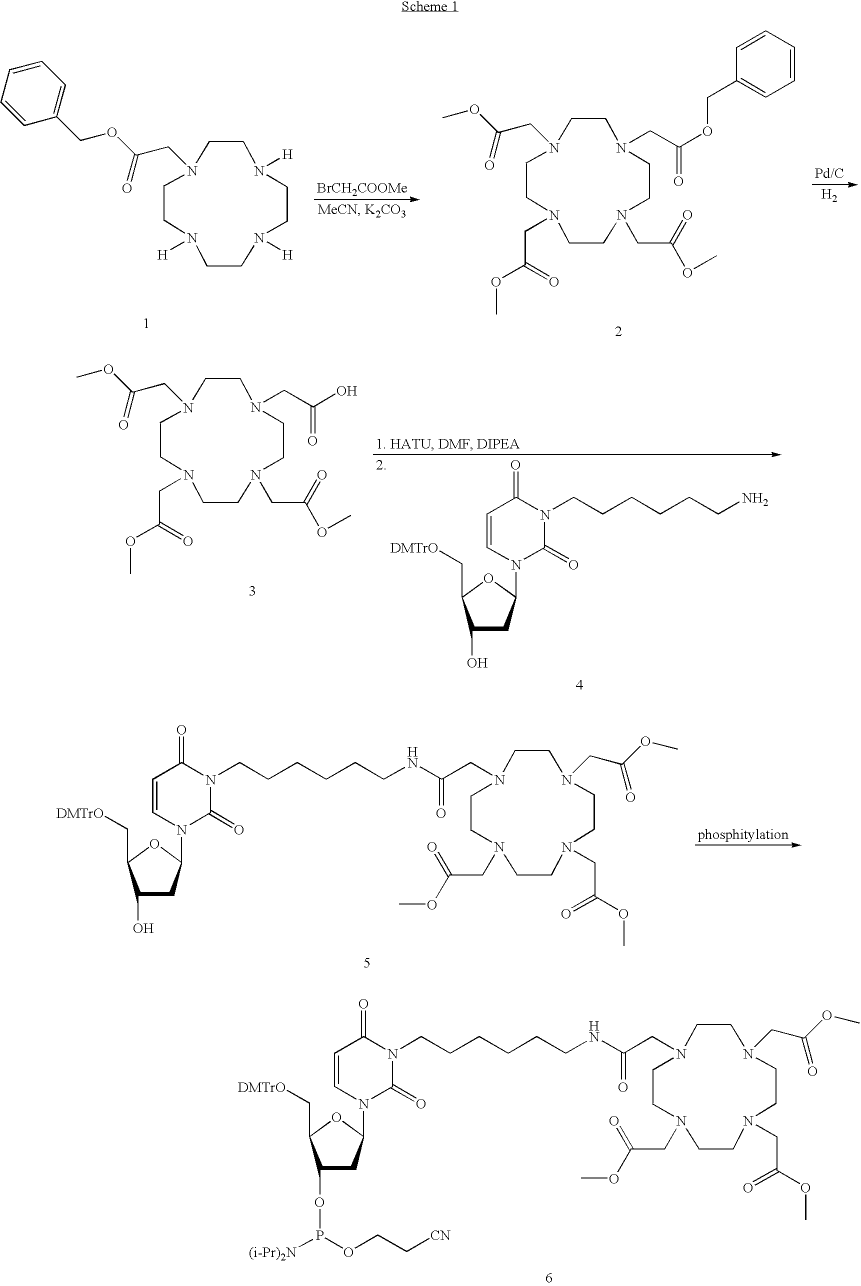

1. A labeling reactant of formula (I) suitable for labeling of an oligonucleotide using solid-phase synthesis 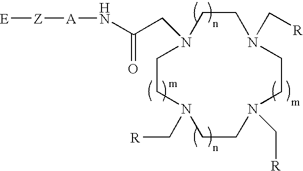
(I)
wherein,
-A- is a linker;
R is —COOR′ or CONHR′ where R′ is an alkyl of 1 to 4 carbon atoms, phenyl or benzyl, which phenyl or benzyl is substituted or unsubstituted;
m and n are independently 0, 1 or 2;
Z is a bridge point and is absent or is a radical of a purine base or a pyrimidine base or a 7-deazapurine base or any other modified base suitable for use in the synthesis of modified oligonucleotides, said base being connected to E via either i) a hydrocarbon chain, which is substituted with a protected hydroxyethyl or hydroxymethyl group, or via ii) a furan ring or pyrane ring or any modified furan or pyrane ring, suitable for use in the synthesis of modified oligonucleotides; and
E is either a phosphorylating moiety 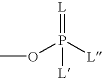
where L is absent or is O or S;
L′ is H, L′″CH₂CH₂CN or L′″Ar, where Ar is phenyl or its substituted derivative, where the substituent is nitro or chlorine, and L′″ is O or S;
L″ is O⁻, S⁻, Cl, N(i-Pr)₂; or
E is a solid support tethered to Z via a linker arm, which is the same as or different from the linker -A- as defined above.

2. The labeling reactant according to claim 1 wherein the linker -A- is formed from one to ten moieties, each moiety being selected from the group consisting of phenylene, alkyl containing 1-12 carbon atoms, ethynediyl (—C≡C—), ethylenediyl (—C═C—), ether (—O—), thioether (—S—), amide (—CO—NH— and —NH—CO— and —CO—NR″ and —NR″—CO—), carbonyl (—CO—), ester (—COO— and —OOC—), disulfide (—SS—), diaza (—N═N—) and tertiary amine (—NR″—), where R″ represents an alkyl containing less than 5 carbon atoms.

3. The labeling reactant according to claim 1 wherein Z is a radical of any of the bases thymine, uracil, adenine, guanine or cytosine, deazaadenine or deazaguanine and said base is connected to E via either i) a hydrocarbon chain, which is substituted with a protected hydroxyethyl or hydroxymethyl group, or via ii) a furan ring having a protected hydroxymethyl group in its 4-position and optionally a hydroxyl, protected hydroxyl, halogen or modified hydroxyl group in its 2-position.

4. The labeling reactant according to claim 3 wherein Z is a radical of adenine, cytosine or 7-deazaadenine where the exocyclic amino group is protected with a protecting group.

5. The labeling reactant according to claim 4 wherein the protecting group is a benzoyl group.

6. The labeling reactant according to claim 3 wherein Z is a radical of guanine or 7-deazaguanine where the exocyclic amino group is protected with a protecting group.

7. The labeling reactant according to claim 6 wherein the protecting group is an isobutyryl group.

8. The labeling reactant according to claim 3 wherein the furan ring is derived from 2-deoxy-D-ribose.

9. The labeling reactant according to claim 3 wherein E-Z-A is selected from the group consisting of the nine structures shown below: 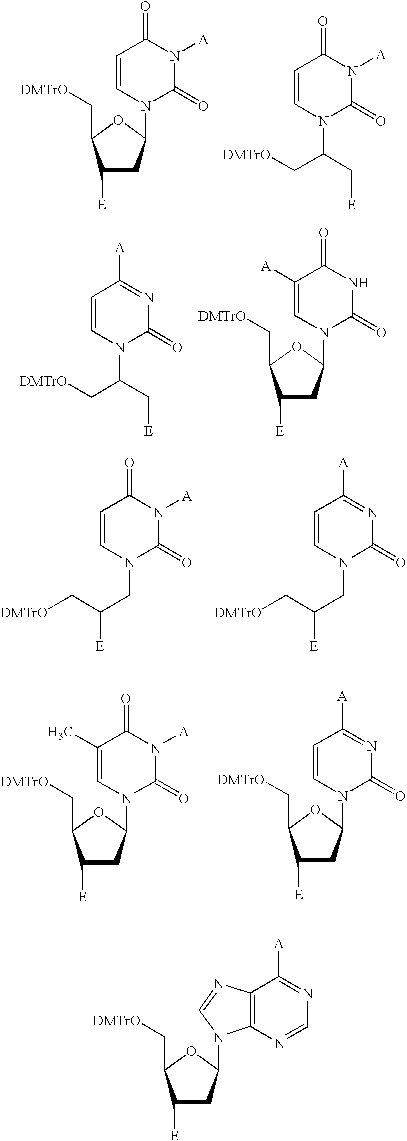
wherein DMTr is dimethoxytrityl.

10. The labeling reactant according to claim 1 wherein L is absent, L′ is OCH₂CH₂CN and L″ is N(i-Pr)₂.

11. An oligonucleotide conjugate synthesized using a oligonucleotide labeling reactant disclosed in claim 1.

12. The labeling reactant according to claim 2 wherein Z is a radical of any of the bases thymine, uracil, adenine, guanine or cytosine, deazaadenine or deazaguanine and said base is connected to E via either i) a hydrocarbon chain, which is substituted with a protected hydroxyethyl or hydroxymethyl group, or via ii) a furan ring having a protected hydroxymethyl group in its 4-position and optionally a hydroxyl, protected hydroxyl, halogen or modified hydroxyl group in its 2-position.

13. The labeling reactant according to claim 2 wherein L is absent, L′ is OCH₂CH₂CN and L″ is N(i-Pr)₂.

14. The labeling reactant according to claim 3 wherein L is absent, L′ is OCH₂CH₂CN and L″ is N(i-Pr)₂.

15. The labeling reactant according to claim 4 wherein L is absent, L′ is OCH₂CH₂CN and L″ is N(i-Pr)₂.

16. The labeling reactant according to claim 5 wherein L is absent, L′ is OCH₂CH₂CN and L″ is N(i-Pr)₂.

17. The labeling reactant according to claim 6 wherein L is absent, L′ is OCH₂CH₂CN and L″ is N(i-Pr)₂.

18. The labeling reactant according to claim 7 wherein L is absent, L′ is OCH₂CH₂CN and L″ is N(i-Pr)₂.

19. The labeling reactant according to claim 8 wherein L is absent, L′ is OCH₂CH₂CN and L″ is N(i-Pr)₂.

20. The labeling reactant according to claim 9 wherein L is absent, L′ is OCH₂CH₂CN and L″ is N(i-Pr)₂.
